(19) United States
(12) Patent Application Publication
(10) Pub. No.: US 2015/0209194 A1
(43) Pub. Date: Jul. 30, 2015

(54) HYGIENIC ARTICLE

(71) Applicant: Ian A. HEYMAN, Pataskala, OH (US)

(72) Inventor: Ian A. HEYMAN, Pataskala, OH (US)

(21) Appl. No.: 14/603,824

(22) Filed: Jan. 23, 2015

Related U.S. Application Data
(63) Continuation-in-part of application No. PCT/US2013/052297, filed on Jul. 26, 2013.
Application No. PCT/US2013/052297 is a continuation-in-part of application No. 13/558,818, filed on Jul. 26, 2012, now Pat. No. 8,961,482.
(60) Provisional application No. 61/512,709, filed on Jul. 28, 2011.
(60) Provisional application No. 62/043,941, filed on Aug. 29, 2014.

(51) Int. Cl. A61F 13/471; A61F 5/453; A61F 5/451

(52) U.S. Cl. CPC A61F 13/471; A61F 5/451; A61F 5/453

(57) ABSTRACT

A hygienic article for a male is disclosed. The hygienic article includes a water resistant tube having an open first end and a closed second end, wherein the first end defines an opening configured to receive a limp penis within an interior of the tube, and an absorbent material disposed within a portion of the interior of the tube configured to absorb urine, wherein the second end is configured to provide radial support to the tube to reduce axial force tending to remove the hygienic article from the penis.

### CROSS-REFERENCE TO RELATED APPLICATIONS

This application is a continuation-in-part of International Application PCT/US2013/052297, filed Jul. 26, 2013, which claims priority to U.S. non-provisional patent application Ser. No. 13/558,818 filed Jul. 26, 2012, which claims priority to U.S. Provisional Patent Application No. 61/512,709, filed Jul. 28, 2011. This application also claims priority to U.S. Provisional Patent Application No. 62/043,941, filed Aug. 29, 2014. The contents of all of the foregoing applications are incorporated herein by reference in their entirety.

### BACKGROUND AND SUMMARY

This present disclosure is related to a hygienic article for males.

Incontinence in a man can arise for many reasons and at any age. For example vehicular accidents, sporting accidents, and any number of medical conditions can give rise to incontinence. Incontinence can also be a product of age, whereby a man's prostate becomes enlarged, surgery effects the nerves serving the bladder, or the man develops Alzheimer's disease or dementia. Incontinence in a man may present by the sporadic or continual trickling of urine from the penis, or may result in spontaneous release of the entire contents of the bladder.

In a hospital or nursing home catheters may be employed to direct the urine into a catchment bag for later disposal. Alternatively the catheter may be used to prevent the continuous or spontaneous release of urine until the patient or resident goes to the bathroom. Catheters are invasive to use and have been responsible for introducing infection, creating undesired health risks for an incontinent man.

Current substitutes to catheters designed to aid an incontinent man suffer from being conspicuous and bulky and may make noise when moving about. Thus they are embarrassing for males of any age to wear, and hamper mobility. Such incontinence products further suffer from not being tailored to target the specific area in which urine exits the body. Throughout the day a man's penis may not remain positioned such that urine may be collected into an incontinence pad or a diaper. This can cause embarrassment and is unhygienic. Furthermore the adult diaper or pad can be easily replaced only when a person is standing. The care giver will either have to move the patient or resident into a standing position, a task made especially challenging when a patient is also infirm and immobile, or continually roll and move the patient who is lying in bed to position the incontinence product underneath him. Among other problems, these obstacles may result in the incontinence diaper or pad being incorrectly positioned on the patient, or it may result in the incontinence diaper or pad being changed less frequently than desired. Additionally, the size of the conventional incontinence pad or diaper means that it will be in contact with areas of skin other than the person's penis. This provides for bodily fluids, such as urine, to be in contact, intermittently or continually, with a much greater area of the man's body, which is unsanitary and can cause skin irritation. Additionally, some prior male incontinence products have employed a clamping mechanism formed of plastic bars that pinch the penis to secure the product to the user. Such products have caused discomfort for user and also restrict blood flow in the penis often requiring that the product be periodically removed.

Using conventional incontinence products may have adverse psychological and emotional effects on the male wearer. For example the adult nappy or diaper may make the incontinent patient feel that he is being treated like a young child. Also, many incontinence products, such as the incontinence guard worn similarly to a woman's sanitary napkin, are associated with and have the appearance of products that a female might wear, causing the incontinent man to feel emasculated. The emotional and psychological effects associated with incontinence are compounded by the products currently available on the market. A male suffering from being incontinent is compelled to accept these emotional and psychological problems or is dissuaded from using the products altogether.

Male underwear garments are well known, such as, briefs, boxer shorts, boxer briefs, bikini briefs, thongs, jock straps, and variants thereof. Virtually every variant of male underwear includes a pouch for holding both the penis and scrotum. This construction may overly constrict or crowd the penis and scrotum, causing a reduction in blood flow to the genitals and potentially impairing, temporally or permanently, the function of the testicles. The over-crowding may also cause entanglements, sweating, sticking, irritation, abrasions, and misalignments.

The man who desires to be less confined by the constraints of the currently available underwear must forgo all protection and hygiene. This allows exposure to bodily fluids such as sweat and urine, which can cause damage to the skin due to excess moisture on the skin and also from irritation caused by the chemical makeup of the bodily fluids. Further harm to skin can arise from the friction between the man's penis and the inside surface of any clothing article the man may be wearing or the man's own thighs and groin area, and may exacerbate the irritant effects of sustained exposure to bodily fluids.

In view of the limitations of presently available products, there is a need for a hygienic article which can be easily and conveniently replaceable, and which offers the protection required by the incontinent male without the adverse emotional, psychological, and physical effects associated with products currently available. There is also a need for an article for a male to wear to overcome the problems with conventional underwear while offering hygienic protection.

Presently disclosed is a hygienic article for a male comprising a tube having a first end adapted to receive a limp penis and a closed second end, the tube being formed of breathable material, a retaining portion adjacent the first end of the tube adapted to retain the hygienic article on the penis without substantially constricting blood flow in the penis, and an absorbent material extending at least within the second end of the tube adapted to absorb fluids. The absorbent material may be provided with the tube or may be separately provided. In one embodiment, the absorbent material may be replaced while the remainder of the hygienic article is reused.

The retaining portion may be adapted in many ways to retain the hygienic article on the penis. For example the retaining portion may comprise an elasticized band, or it may have elasticized thread woven into the breathable material of the tube adjacent the first end, both adapted to extend around the penis and to retain the hygienic article on the penis. Alternatively the retaining portion may comprise at least one elastic tie adapted to extend around the penis and to retain the hygienic article on the penis. Further the retaining portion may comprise at least one adhesive strip adapted to be secured to the penis or pelvic area to retain the hygienic article on the penis. The retaining portion may comprise silicone beading configured to retain the hygienic article on the penis.

Also disclosed is a hygienic article for a male comprising a water resistant tube having an open first end and a closed second end, wherein the first end defines an opening configured to receive a limp penis within an interior of the tube, and an absorbent material disposed within a portion of the interior of the tube configured to absorb urine, wherein the second end is configured to provide radial support to the tube to reduce axial force tending to remove the hygienic article from the penis.

In some embodiments, the tube comprises a layered composite material.

Further disclosed is another embodiment of a hygienic article for a male, wherein the layered composite material comprises a multi-layer waterproof outer covering, the absorbent material, an acquisition distribution layer configured to transport urine away from the penis and into the absorbent material, and a topsheet configured to be in contact with a penis.

Also disclosed is another embodiment of a hygienic article for a male, wherein the multi-layer waterproof outer covering is an elasticized non-woven material.

In some embodiments, the second end comprises at least two flaps, wherein the at least two flaps are integral with the tube, folded and physically connected to each other to form the closed second end of the tube,

Further disclosed is another embodiment wherein the second end comprises pleating.

Also disclosed is another embodiment wherein the pleating is a box pleat.

Further disclosed is another embodiment wherein the second end further comprises an external flap configured to provide structural support to maintain the shape of the second end while the hygienic article is in use.

Also disclosed is an embodiment wherein the exterior flap is perpendicular to a longitudinal axis extending between the first end and the second end of the tube.

Also disclosed is another embodiment wherein the second end is integrally formed with the tube, and the second end further comprises a semi-circular cross section.

Further disclosed is another embodiment wherein the second end further comprises an interior fold.

Further disclosed is another embodiment wherein the tube further comprises two side portions extending between the first end and the second end, and the two side portions each comprise an interior fold along at least a portion of the length of the side portions.

In some embodiments, the tube is formed from two separate pieces of layered material physically connected around at least a portion of the perimeter of the pieces to form the hygienic article.

Also disclosed in some embodiments, the tube has a rectangular cross section.

In some embodiments, the water resistant tube further comprises a top portion, a bottom portion, and at least two side portions, in which the top portion and the bottom portion are parallel with respect to each other, the at least two side portions are parallel with respect to each other, and the first end and second end are positioned parallel with respect to each other.

Also disclosed in some embodiments is a hygienic article for a male further comprising at least one retaining strap located adjacent the first end.

In some embodiments, disclosed is a retaining strap further comprises a retaining material, wherein the retaining material can be directly releasably attached to the tube.

Also disclosed is a hygienic article for a male, further comprising at least two side portions, in which the at least two side portions are parallel with respect to each other, and the second end is positioned perpendicular to the at least two side portions.

### BRIEF DESCRIPTION OF THE DRAWINGS

FIG. 1 is a perspective view of a first embodiment of a hygienic article for a male.

FIG. 2 is a perspective view of another embodiment of a hygienic article for a male.

FIG. 3 is a perspective view of another embodiment of a hygienic article for a male.

FIG. 4 is a perspective view of another embodiment of a hygienic article for a male.

FIG. 5 is a perspective view of another embodiment of a hygienic article for a male.

FIG. 6 is a cross-sectional view of another embodiment of a hygienic article for a male.

FIG. 7 is a perspective view of an embodiment of a hygienic article for a male, rolled up.

FIG. 8 is a side view of another embodiment of a hygienic article for a male.

FIG. 9 is a cutaway view of another embodiment of a hygienic article for a male.

FIG. 10 is a side perspective view of another embodiment of a hygienic article for a male.

FIG. 11 is a front perspective view of another embodiment of a hygienic article for a male.

FIG. 12 is an angled orthogonal view of another embodiment of a hygienic article for a male.

FIG. 13 is an angled orthogonal view of another embodiment of a hygienic article for a male.

FIG. 14 is an angled orthogonal view of another embodiment of a hygienic article for a male.

FIG. 15 is an angled orthogonal view of another embodiment of a hygienic article for a male.

FIG. 16 is an angled orthogonal view of another embodiment of a hygienic article for a male.

FIG. 17 is an angled orthogonal view of another embodiment of a hygienic article for a male.

FIG. 18 is a cross-sectional view of the interior of a hygienic article for a male.

FIG. 19 is a cross-sectional view of another embodiment of the interior of a hygienic article for a male.

FIG. 20 is a top view of one embodiment of an unfolded hygienic article for a male.

FIG. 21 is a top view of another embodiment of an unfolded hygienic article for a male with round ends.

FIG. 22 is a top view of a bottom portion of another embodiment of a hygienic article for a male and a cut away of the material used.

FIG. 23 is a top view of a top portion of hygienic article for a male.

FIG. 24 is a angled orthographical view of a top and bottom portion being formed together to create a hygienic article for a male.

FIG. 25 is a top view of another embodiment of an unfolded hygienic article for a male having a vertical folding line and various locations of absorbent material.

FIG. 26 is a top view of another embodiment of an unfolded hygienic article for a male with sides that are angled.

FIG. 27 is a top view of another embodiment of an unfolded hygienic article for a male with an additional piece of fabric in the center fold.

FIG. 28 is a top view of a piece of continuous material in which an unfolded hygienic article may be cut from without waste

FIG. 29 is a top view of another embodiment of a piece of continuous material in which an unfolded hygienic article may be cut from without waste

### DETAILED DESCRIPTION OF THE DRAWINGS

Presently disclosed is an easily replaced hygienic article for a male providing comfort and protection. The hygienic article may also protect against the effects of incontinence or unintended urination. The hygienic article may be easily applied regardless of the position or mobility of the man or the man's penis. The hygienic article for a male also allows the male wearer to be free of the confines of conventional underwear while still being afforded hygienic protection. Furthermore, the hygienic article for a male may be configured such as to be easily disposable.

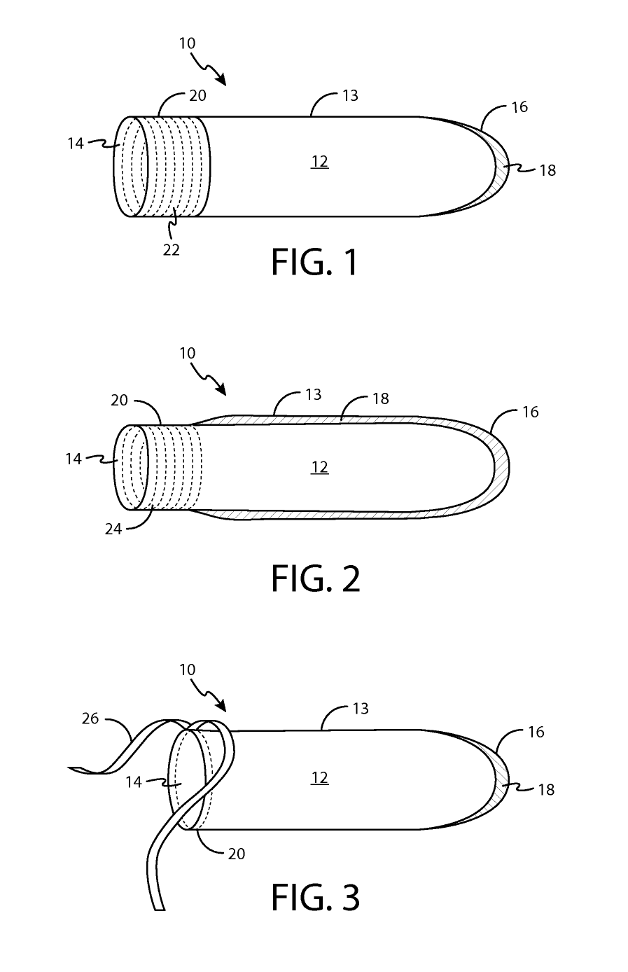

Referring generally to FIGS. 1 through 7, embodiments of a hygienic article for a male are disclosed. In one embodiment, the hygienic article comprises a tube having a first end adapted to receive a limp penis and a closed second end, where the tube is formed of breathable material. The hygienic article also includes a retaining portion adjacent the first end of the tube adapted to retain the hygienic article on the penis without substantially constricting blood flow in the penis, and an absorbent material extending at least within the second end of the tube adapted to absorb fluids. The absorbent material may be part of the tube or may be separately provided. In some embodiments, the absorbent material may be replaceable within the hygienic article.

Referring to FIG. 1, one embodiment of a hygienic article 10 is illustrated. The hygienic article 10 has a tube 12. The tube 12 has a first end 14 adapted to receive a limp penis and a closed second end 16. The tube 12 is formed of breathable material 13. The hygienic article 10 also has a retaining portion 20 adjacent the first end 14 of the tube 12 adapted to retain the hygienic article 10 on the penis without substantially constricting blood flow in the penis, and an absorbent material 18 extending at least within the second end of the tube 12 adapted to absorb fluid. The first end 14 adapted to receive a limp penis may accommodate multiple size penises. In addition, it is contemplated that the hygienic article 10 will be produced in multiple sizes, in order to fit the individual needs of the wearer. In one embodiment, a portion of the tube 12 may be formed of elastic material to assist in retention of the hygienic article 10. The portion of the tube 12 formed of elastic material may be adjacent the first end of the tube 12. In another embodiment, the entire tube 12 may be formed from elastic material to form an elasticized tube.

In use, the absorbent material 18 may absorb fluids, such as urine. In various embodiments, the absorbent material 18 is adapted to absorb a desired amounts of fluid, such as, for example, only small amounts of urine or, in an alternative embodiment, an entire bladder-full of urine. The absorbent material 18 may be confined to a specific portion of the hygienic article 10, such as within the second end 16. Alternatively, the absorbent material 18 may extend substantially between the second end 16 and the first end 14, or the absorbent material 18 may extend throughout the entire length of the tube 12. Moreover, the absorbent material 18 may extend only partially around the circumference of the tube 12, or, as shown in FIG. 1, extend substantially around the circumference of the tube 12. In a further embodiment, described in detail below, the absorbent material 18 may be integral with the tube 12, and also integral with the breathable material 13. The absorbent material 18 may be made from natural materials, synthetic materials, or combinations of natural and synthetic materials, such as, cotton, hemp, sponge, wool, cork, synthetic sponge, polyester or polyester mixes, neoprene, plastics, synthetic cork, or fleece materials. The breathable material 13 may also be made of natural materials, synthetic materials, or combinations of natural and synthetic materials, such as, rayon, nylon, spandex, cotton, hemp, leather, lycra, elastics, microfiber or wool, in addition to the materials previously mentioned. In one embodiment, the breathable material 13 comprises tubular stretch net material made from high quality nylon and rubber mix, and the absorbent material 18 comprises a sanitary towel or similar product material.

In the embodiment illustrated in FIG. 1, the retaining portion 20 is a band of elasticized thread 22 interwoven into the tube 12, extending substantially around the circumference of the hygienic article 10. The retaining portion 20 extends around the penis and retains the hygienic article 10 on the penis without substantially constricting blood flow in the penis. Alternatively, elasticized thread 22 may be intermittently interwoven into the tube 12, around the circumference of the hygienic article 10. In one embodiment, the retaining portion 20 is positioned at the first end 14 of the tube 12. In other embodiments, the retaining portion 20 may extend from the first end 14 of the tube. In yet other embodiments, the retaining portion 20 may be integrated with the tube 12 and may extend at least partially towards the second end 16 to achieve a desired retention force on the penis.

An incontinent male is able to wear the hygienic article 10 without the adverse emotional and psychological effects that are associated with adult nappies, diapers and incontinence pads. Furthermore, the hygienic article 10 may be more effectively retained on the penis in the proper position to received urine. The hygienic article 10 thus aids in preventing urine from coming into contact, temporarily or permanently, with other areas of the body, such as the upper thighs, buttocks, abdomen or scrotum.

Another embodiment of a hygienic article 10 is illustrated in FIG. 2. As shown in FIG. 2, the hygienic article 10 includes a tube, such as an elasticized tube 12 with a first end 14 and a closed second end 16 opposite the first end 14. The first end 14 of the elasticized tube 12 is adapted to receive a limp penis. The elasticized tube 12 is formed of breathable material 18, and the first end 14 of the elasticized tube 12 includes a retaining portion 20 adapted to apply sufficient pressure to retain the hygienic article on the penis without substantially constricting blood flow. For example, the hygienic article 10 may be worn continuously for at least 3 hours, at least 6 hours, or at least 12 hours, or more. The absorbent material 18 which absorb fluids as discussed above, extends between adjacent the first end 14 and the closed second end 16. As shown in FIG. 2, the retaining portion 20 includes an elasticized band 24 adapted to extend around the circumference of the penis to retain the hygienic article 10 on the penis. In yet another embodiment, at least a portion of the second end 16 of the elasticized tube 12 comprises absorbent material 18 adapted to absorb fluids. For example, the absorbent material 18 may be integral with the second end 16 of the elasticized tube 12.

In another embodiment, a hygienic article 10 includes an elasticized tube 12 having a first end 14 adapted to receive a limp penis and a closed second end 16 opposite the first end 14, where the elasticized tube 12 is formed of breathable material 13. The hygienic article 10 also includes a base portion adjacent the first end of the elasticized tube 12 adapted to apply sufficient pressure to retain the hygienic article 10 on the penis without substantially constricting blood flow. The hygienic article 10 also includes a tip portion adjacent the second end 16 of the elasticized material forming an enclosed end of the hygienic article 10, where at least a portion of the tip portion comprises an absorbent material 18 adapted to absorb fluids. The base portion of the hygienic article 10 may include elasticized thread or an elasticized band as discussed above. The base portion and tip portion may be integral with the elasticized tube 12 or may be formed separately and attached to the elasticized tube during manufacturing of the hygienic article 10.

In a further embodiment, as shown in FIG. 3, a hygienic article 10 is provided that includes an elasticized tube 12 having a first end 14 adapted to receive a penis and a closed second end 16, where the elasticized tube 12 is formed of a breathable material 13. The hygienic article 10 has a retaining portion 20 adjacent the first end 14 of the elasticized tube 12 adapted to retain the hygienic article 10 on the penis without substantially constricting blood flow, and an absorbent material 18 extending at least within the second end 16 of the elasticized tube 12 adapted to absorb fluids. As shown in FIG. 3, the retaining portion 20 includes one or more ties 26 adapted to extend around the circumference of the penis to retain the hygienic article 10 on the penis. The one or more ties 26 may be elastic and may be tied into a knot, such as the bow illustrated in FIG. 3, to secure the hygienic article 10 onto the penis.

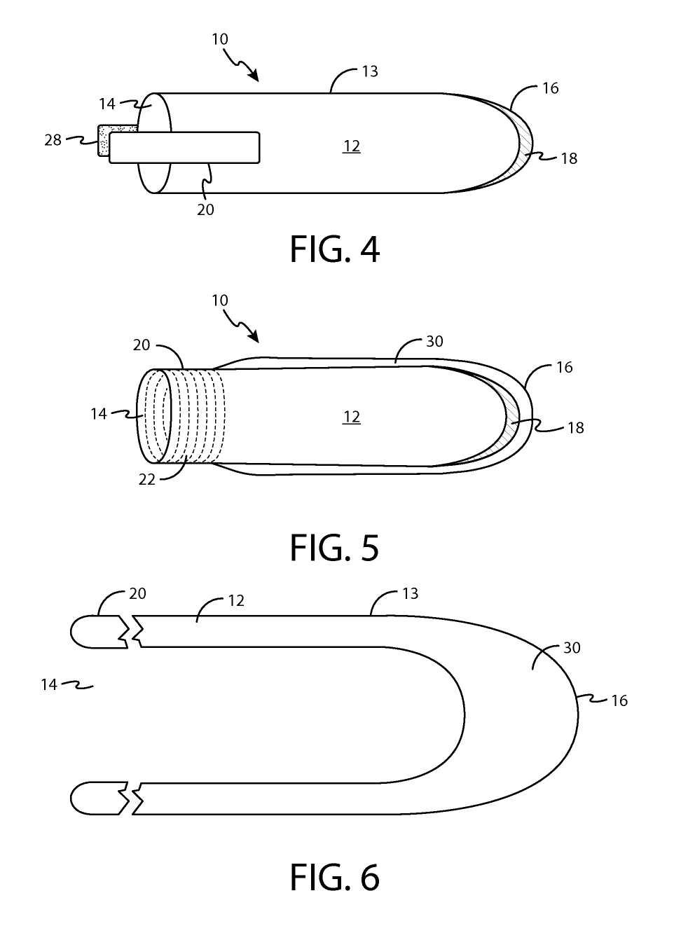

In yet another embodiment, the retaining portion 20 may optionally include at least one adhesive strip 28 adapted to secure the hygienic article 10 to the wearer. As illustrated in FIG. 4, the hygienic article 10 has an elasticized tube 12 with a first end 14 and a closed second end 16 opposite the first end 14. The retaining portion 20 including the at least one adhesive strip 28 is adapted to retain the hygienic article 10 on the penis without substantially constricting blood flow. The at least one adhesive strip 28 is secured to the penis or pelvic region of the wearer to retain the hygienic article 10 on the penis. Adhesive strips 28 provide for minimal compression around the base of the penis allowing the hygienic article 10 to be worn for an extended time until needing to removed or replaced.

In a further embodiment, the retaining portion 20 may include at least one strip of silicone beading adapted to retain the hygienic article 10 on the penis. Silicone beading may be applied directly to the elasticized tube 12 at the retaining portion 20, or may be attached to a further piece of elasticized fabric which may be attached to the retaining portion of the hygienic article 10. The at least one strip of silicone beading may extend around the circumference of the retaining portion or may be attached intermittently around the circumference of the retaining portion.

In yet another embodiment, the hygienic article 10 further comprises padding 30 adapted to enlarge the external dimensions of the hygienic article 10. As shown in FIG. 5, padding 30 may extend substantially between the first end 14 and the second end 16 of the elasticized tube 12. Alternatively the hygienic article for a male 10 may be configured such that the padding 30 extends substantially within the second end 16, and/or on one side of the elasticized tube 12. The hygienic article 10 including padding 30 may enhance the outer aesthetic dimensions of the penis, having a similar esthetic effect as padded bras for women. Alternatively, the hygienic article 10 including padding 30 may provide additional support or protection for the penis when the hygienic article is worn in lieu of conventional underwear.

In yet another embodiment, the elasticized tube 12 of the hygienic article 10 comprises padding 30 adapted to enlarge the external dimensions of the hygienic article 30. The padding 30 may be constructed from the same material as the elasticized tube 12, such that the elasticized tube 12 is integral with the padding 30. In a further embodiment, not shown, the padding 30 and the absorbent material 18, may be constructed of the same material as the elasticized tube 12, such that the elasticized tube 12 is integral with both the absorbent material 18, and the padding 30. The padding 30 may be made from natural or synthetic materials such as cotton, wool, leather, cork, sponge, synthetic sponge, polyester or polyester mixes, plastics, neoprene, or fleece material. Constructing the various components of the hygienic article 10 of the same material may reduce manufacturing costs. Additionally, integrating the components of the hygienic article 10 may reduce the need for sewing and the number of seams, thereby reducing the possibility of leakage and improving the performance of the hygienic article 10.

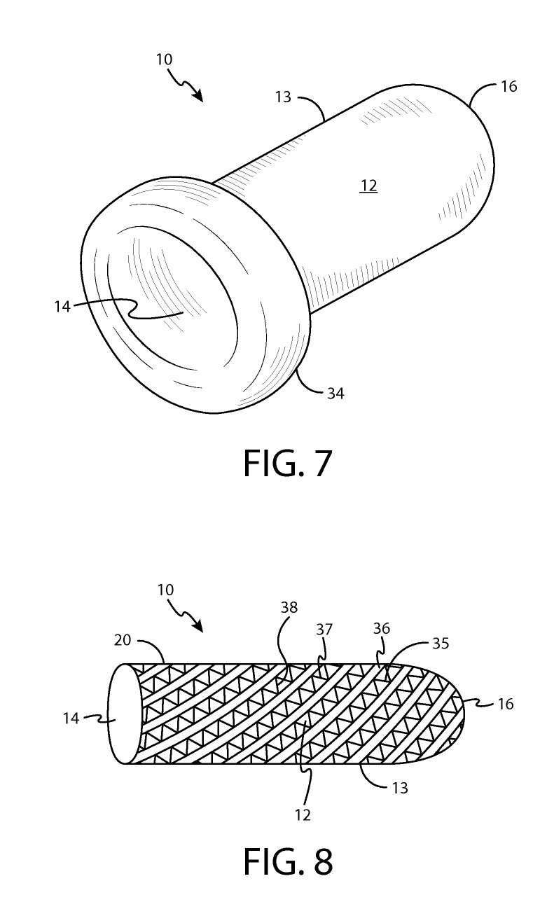

During use, the hygienic article 10 may be provided in a rolled configuration such as illustrated in FIG. 7 to assist with application of the hygienic article 10. As illustrated, the elasticized tube 12 may be rolled to form a rolled portion 34. The hygienic article 10 may then be placed on the tip of the man's penis so that the man's penis enters the hygienic article 10 through an opening 17 within the rolled portion 34 and the tip of the penis is positioned adjacent the closed second end 16. The rolled portion 34 may then be rolled down the penis so that the elasticized tube 12 substantially covers the penis with the first end 14 resting near the base of the penis. The hygienic article 10 shown in FIG. 7 provides for an easy application of the hygienic article 10 and ease of replacement when an incontinent male is in a seated or lying position, thus reducing the need to reposition the patient or resident as would be the case with traditional nappies, diapers or pads. Hygienic article 10 may be stored and packaged in the rolled configuration, as described above, such as to allow the user to open the packaging containing the hygienic article 10 and apply the hygienic article without first rolling it. Furthermore, such configuration may aid in the shipping and storage of hygienic article 10. In another embodiment, the hygienic article 10 may be adapted for repeated use. The hygienic article 10 may be washable or sanitized between uses, and the absorbent material may be replaceable.

The elasticized tube 12, of hygienic article 10, may further comprise a waterproof outer coating (not shown) adapted to prevent the movement of fluid across the waterproof outer coating, therefore preventing the ingress or egress of fluid to and from the hygienic article 10. The waterproof coating may be breathable, and may be made from plastics, cellulose-based compounds, rubber, or other waterproof materials. In an alternative embodiment, the waterproof outer coating may be integral with the elasticized tube 12.

A hygienic article for male may be configured to be particularly useful as a decorative fashion accessory or as a novelty item. The hygienic article 10 adapted for use as a fashion accessory or novelty item may comprise an elasticized tube 12 having a first end 14 adapted to receive a limp penis and a closed second end 16 opposite the first end 14, the elasticized tube 12 being formed of breathable material 13, where the first end 14 of the elasticized tube 13 comprises a retaining portion 20 adapted to apply sufficient pressure to retain the hygienic article 10 on the penis without substantially constricting blood flow, and where the elasticized tube 13 comprises padding 30 adapted to enlarge the external dimensions of the hygienic article 10, thereby increasing the apparent size of the penis. Furthermore, in circumstances where the wearer of the hygienic article 10 prefers to wear pants at a lower position, hygienic article 10, comprising padding 30, aids in supporting the pants in the desired position. The hygienic article 10 may further comprise decoration on the outer surface of the elasticized tube 12. Such decoration may comprise of a single color or a pattern to enhance the aesthetic appearance of the hygienic article 10. In another embodiment, the article 10 may be provided without padding to provide a slim protective sheathing for the penis.

FIG. 8 shows a hygienic article for a male 10 comprising an elasticized tube 12 having a first end 14 adapted to receive a limp penis and a closed second end 16, the tube 12 being formed of breathable material 13, a retaining portion 20 adjacent the first end 14 of the tube 12 adapted to retain the hygienic article 10 on the penis without substantially constricting blood flow, where the tube 12 may comprise tubular stretch net material 35. An example of tubular stretch net material is Surgilast® sold by Derma-Science, inc. FIG. 8 shows a possible configuration whereby tubular stretch net material 35 may comprise diagonal main thread 36 positioned in one direction and diagonal support thread 37 positioned in another direction, such that diagonal main thread 36 and diagonal support thread 37 form a diamond pattern. Further, tubular stretch net material may comprise of cross support thread 38 designed to increase the compressive strength of the tube 12 on the penis, without constricting blood flow. Tubular stretch net material 35 may be constructed in many different configurations and achieve the characteristics desired in the elasticized tube 12. Tubular stretch net material 35 may be made from nylon and rubber mix and may be latex-free to prevent reaction with latex-intolerant wearers of hygienic article 10.

Hygienic article 10 may be assembled such that the entire article is woven from a single material. Alternatively, if separate materials are used, or separate sections of the same material are used, the various elements may be attached using different methods. Attachment methods may include weaving, sewing, gluing, or connecting, such as with Velcro®, or any manner which may connect components together.

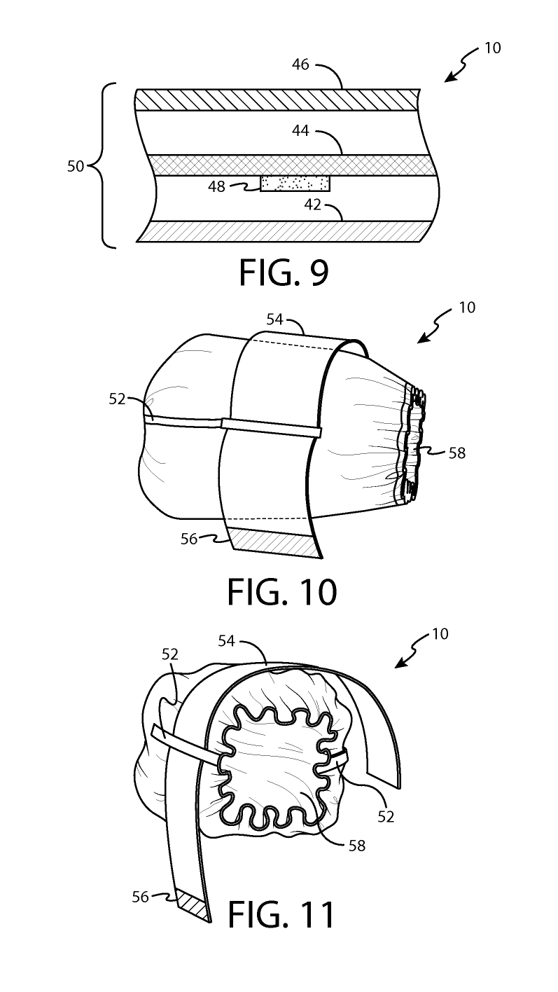

FIGS. 9-11 illustrate another embodiment of the hygienic article 10. FIG. 9 is a side cutaway view showing the various layers of the hygienic article 10. As shown in FIG. 9, the hygienic article includes a first layer 42 adjacent the body, a second intermediate layer 44, and a third layer 46 that serves as the outer cover. Disposed between the first 42 and second 44 (or between the second 44 and third 46) layers is an absorbent core 48, such as a desiccant, hydrophilic compound, or other absorbent material. The first layer 42 is a water permeable surface that provides wicking action to draw liquid from the body towards the second layer 44. The second layer 44 is a distribution layer that receives liquid from the first layer and distributes it about the second layer 44. Liquid in the second layer 44 is absorbed by the absorbent core 48, which draws the liquid from the second layer. The third layer 46 is the outermost layer and is a water resistant, water proof, or water impermeable surface or includes a hydrophobic coating that prevents liquid from leaking from the article 10. The combination of these layers in a sandwiched arrangement constitutes the quilted fabric 50 of the hygienic article. Additionally, in some embodiments, the third layer 46 may be a non-woven tri-laminate material capable of stretching in only one direction, and in other embodiments the material may be capable of stretching in a plurality of directions. Furthermore, in some embodiments the second layer 44, first layer 42, and absorbent layer 48 may also comprise of non-woven material capable of stretching in a number of directions. In other embodiments, some layers may comprise of woven material while other layers comprise of non-woven material. It is to be understood that the combination of these layers may occur in any order and still achieve the goal of the invention.

FIGS. 10-11 illustrate an embodiment of the assembled hygienic article 10. A sheet of the above-described fabric 50 is folded and sealed along seams 52 by a welding process to form a substantially rectangular shaped hygienic article. A strap 54 having a connecting end 56, such as an adhesive or hook-and-loop fastener, is provided along one side of the hygienic device 10. The strap extends around the outside of the hygienic device 10 and attaches to the fabric 50 or itself to securely hold the penis inside the device 10. An opening 58 is provided on one side of the device 10. According to one embodiment, the outer edge of the opening 58 is folded and is mechanically welded by either heat sealing, adhesively sealing, sonic welding, or ultrasonically welding. As the mechanically welded material contracts, the opening 58 closes, providing an opening of a flexible size for a snug fit around the base of the penis. Alternatively, the opening 58 may include an elastic band or other means for snugly fitting to the base of the covered penis.

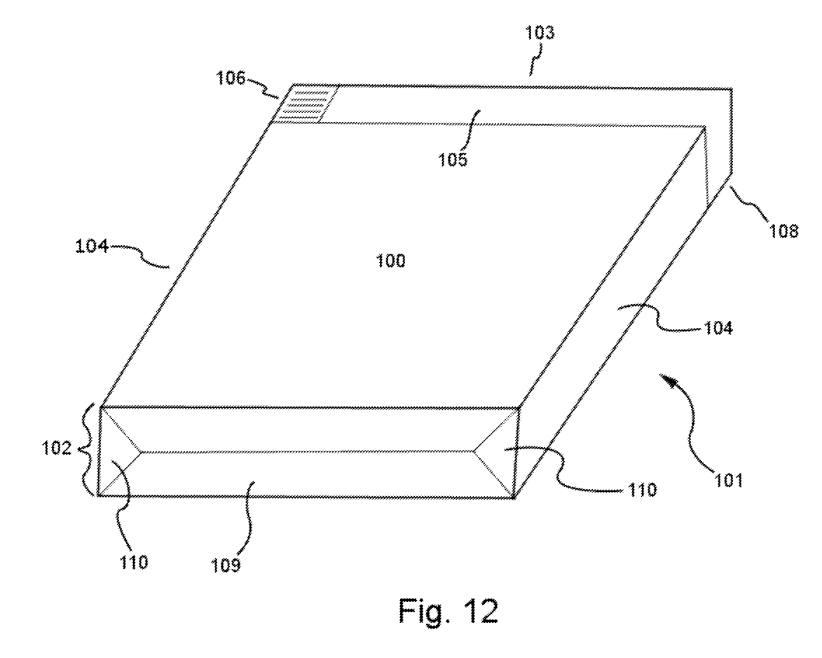

Referring now to FIGS. 12-22, additional embodiments of a hygienic article for a male are disclosed. In various embodiments, the hygienic article for a male includes a water resistant tube having an open first end and a closed second end. The hygienic article also includes an absorbent material disposed within a portion of the interior of the tube configured to absorb urine. The first end of the tube defines an opening configured to receive a limp penis within an interior of the tube. The second end of the tube is configured to provide radial support to the tube to reduce the axial force tending to remove the hygienic article from the penis.

Referring now to FIG. 12, a hygienic article 101 having with a second end 102 closed by flaps 109, 110 is illustrated. In an embodiment, the hygienic articles 101 is a tube 100 formed of a base material extending between a first end 103 for receiving a penis and the closed second end 102. The hygienic article 101 may be configured with side portions 104 defining a substantially rectangular cross-section of the tube. The hygienic article also includes a retaining strap 105.

The retaining strap 105 has an attached end 108, which is attached to the hygienic article, and a retaining material 106 disposed opposite the attached end 108. In one embodiment, the retaining strap 105 is integrally formed with the base material of the hygienic article 101, such that the attached end 108 is integrally connected with the base material. In another embodiment, the retaining strap 105 is formed separate from the base material and connected to the base material during assembly of the hygienic article. In one embodiment, the attached end of the retaining strap 108 is connected near the first end 103 of the hygienic article 101.

The retaining strap 105 is configured to wrap around and compress the dimensions of the first end 103 in order to secure the hygienic article 101 onto the penis. The retaining strap 105 is kept in place by pressing the retaining material 106 onto a portion of the exterior of the base material. In some embodiments, the retaining material 106 may directly engage the base material, however in other embodiments, the retaining material 106 may engage a mating piece of retaining material affixed to the base material . By having a single section of retaining material 106 attached to a single retaining strap 105 integrally formed with the hygienic article 101, the first end of the hygienic article 101 may be reduced as desired by the user to assist in retaining the hygienic article on the user's penis.

In other embodiments, the attached end 108 can be located elsewhere on the hygienic article 101, such as on the base material between the first end and the second end, and still allow the retaining strap 105 to compress the dimensions of the hygienic article 101 in order to secure the hygienic article onto the penis.

As shown in FIG. 12, the second end 102 includes flaps 109, 110. In various embodiments, the flaps 109, 110 are mechanically welded or sewn together to form the closed second end 102. Alternatively, the flaps 109, 110 may be folded from a single piece of base material. In one embodiment, the flaps 109, 110 may connect in such a manner as to form a substantially planer end of the hygienic article. In alternative embodiments, the flaps 109, 110 connect to form an angle either into or away from the interior of the hygienic article 101. In either case, the configuration of the flaps provides support to the second end to maintain the shape of the hygienic article providing space for the user's penis within the hygienic article.

In an exemplary embodiment, the flaps 109, 110 are angled in such a way as to fold into the interior of the hygienic article 101. The angled fold reduces the amount of force acting to remove the hygienic article 101 from the penis while in use.

The components of the hygienic article 101 may be formed integrally—or alternatively—may be formed separately and then mechanically welded and/or sewn together. For example, the hygienic article 101 and may be formed from the quilted fabric 50. In other embodiments, the hygienic article may be formed of a single material that is constructed to provide both the absorbency and moisture resistant portions.

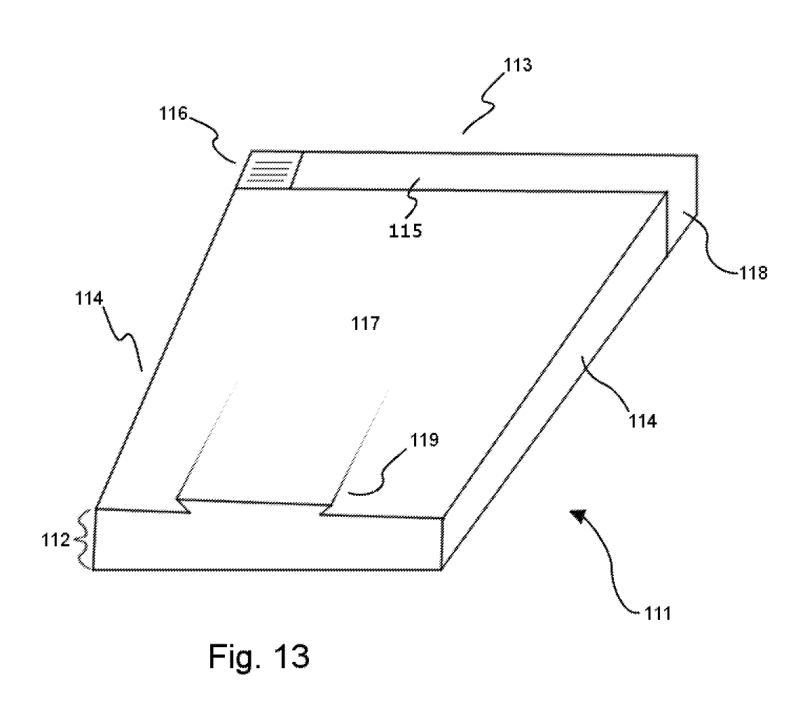

Referring now to FIG. 13, another embodiment of a hygienic article 111 is illustrated that includes a pleated second end 112. The hygienic article 111 is configured as a tube 117, having a first end 113 for receiving a penis, a closed second end 112, and a retaining strap 115 with a retaining material 116. The tube 117 may have a substantially rectangular cross section with side portions 114. In some embodiments the side portions 114 are angled to give the hygienic article 111 a wedge-like shape.

As shown in FIG. 13, the second end 112 of the hygienic article includes at least one pleating 119. The pleating 119 (also referred to as one or more pleats) is formed by folding or sewing the fabric back upon itself. The hygienic article may have one or more pleats. In some embodiments, the pleating 119 provides structural support to maintain the internal volume for the penis within the hygienic article. The pleating 119 may also increase strength and/or flexibility of the hygienic article, providing a more comfortable feel for the user. The pleating 119 may be of any kind of appropriate pleating, such as, accordion pleats, box pleats, fortuny pleats, knife pleats, organ pleats, or fine boxed pleats.

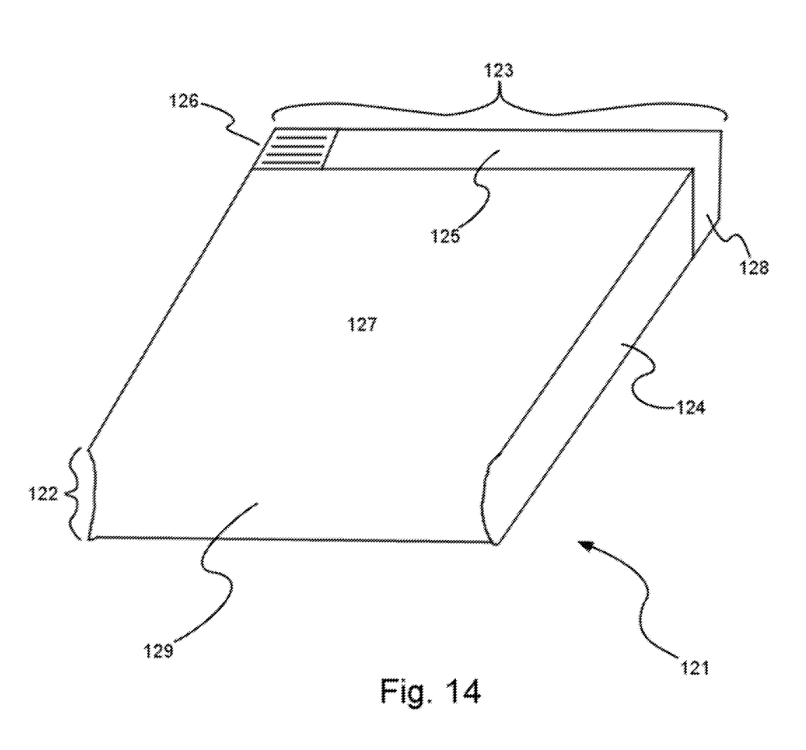

Referring now to FIG. 14, a hygienic article 121 having a rounded end is illustrated. The hygienic article 121 includes a first end 123 for receiving a penis, a closed second end 122, side portions 124, and a retaining strap 125 as previously described. The second end 122 includes a rounded portion 129 that is integrally formed with an upper portion 127 extending toward the first end of the hygienic article. In this embodiment, the rounded portion 129 of the second end 122 bends to form a curve creating the closed second end 122. In some embodiments, the second end has a semi-circular cross section.

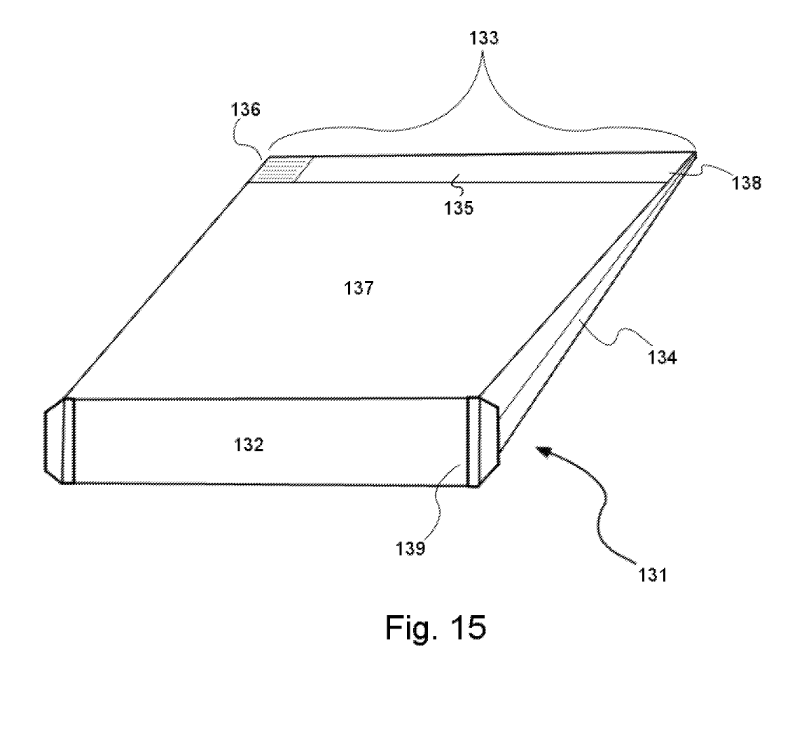

Referring now to FIG. 15, a hygienic article having an external support portion is illustrated. The hygienic article includes a first end 133 for receiving a penis, a second end 132 which can be closed, side portions 134, and a retaining strap 135, as previously described. In addition, the second end 132 includes an external flap 139 that extends outward from the second end 132, and connects a side portion 134, with an upper portion 137 and second end portion 132. The external flap 139 may be formed by mechanical welding or sewing. In embodiments, the external flap 139 is configured to provide structural support to assist in maintaining the shape of the second end 132 while the hygienic article 131 is in use. The external flap further assists in maintaining the shape of the interior of the tube near the second end to reduce the axial force tending to remove the hygienic article from the penis when in use. In some embodiments, such as illustrated in FIG. 15, the external flap is perpendicular to a longitudinal axis that extends between the first end and the second end of the tube. In yet other embodiments, a reinforced seam or other portion of the tube material may be used that does not extend outward from the tube but still assists in maintaining the shape of the second end.

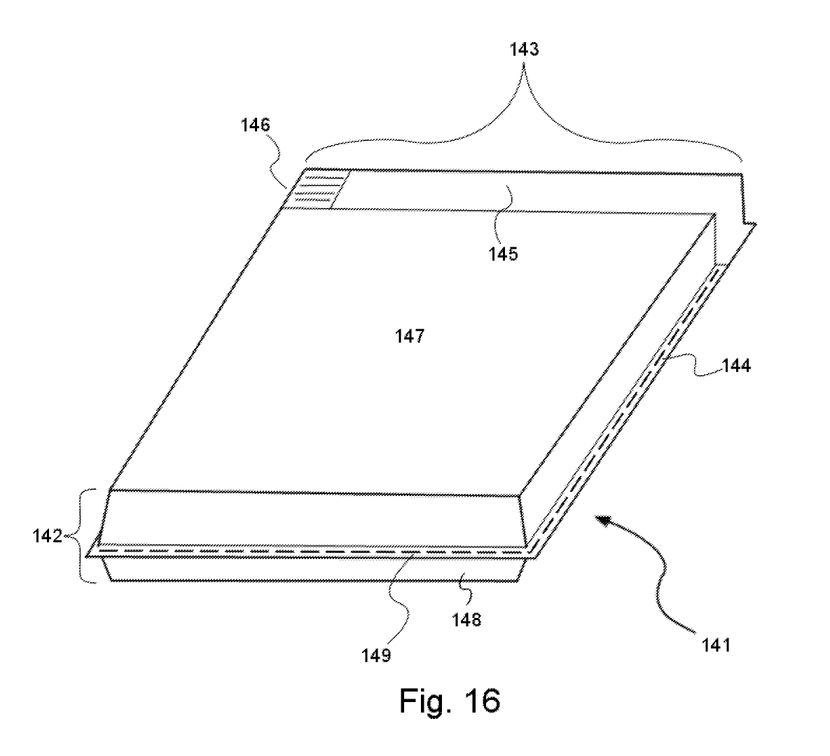

Referring now to FIG. 16, a hygienic article is illustrated that is formed of a top portion 147 and a bottom portion 148 joined by a peripheral connection, extending around at least a portion of the perimeter of the top portion and bottom portion. The hygienic article 141 includes a first end 143 for receiving a penis, a second end 142 which can be closed, side portions 144, and a retaining strap 145. The hygienic article is formed from a top portion 147 overlaid on a bottom portion 148, which are attached by a peripheral connection 149. In some embodiments, the peripheral connection 149 is a mechanical or chemical means of connecting two pieces of fabric material, such as but not limited to: sewing, ultrasonic welding, heat welding, or applying an adhesive material. The peripheral connection 149 can be located around the entire perimeter of the hygienic article 141 except for the opening in the first end, or can be located in discrete locations as needed to maintain the connection between the top portion 147 and bottom portion 148 (such as only in the corners). In the illustrated embodiment, the peripheral connection 149 is located on the side portions 144 and second end 142. In other embodiments, the peripheral connection 149 may be employed anywhere on the hygienic article 149 necessary to provide the structural rigidity required to hold the hygienic article's shape while in use.

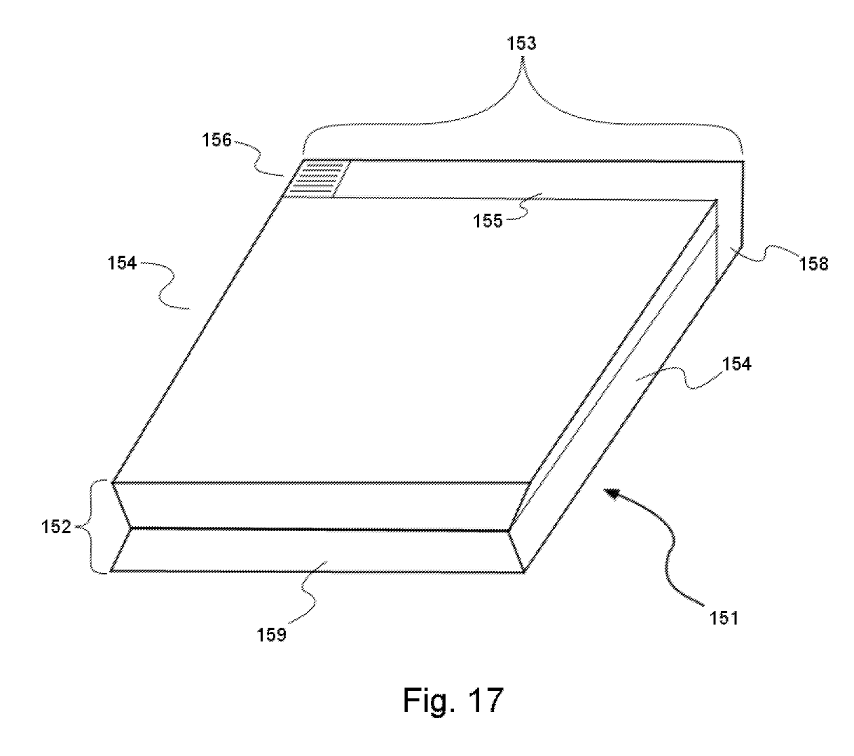

Referring now to FIG. 17, a hygienic article 151 in which the second end includes an interior fold is illustrated. The hygienic article 151 includes a first end 153 for receiving a penis, a second end 152 which can be closed, side portions 154, and a retaining strap 155, as previously described. The hygienic article 151 also includes an interior fold 159 extending substantially along the second end portion 152. Optionally, the interior fold 159 may also extend along at least a portion of the side portions 154 as illustrated. The interior fold 159 may be continuous, or may be formed in several discrete portions along the second end and side portions. The interior fold 159 decreases the interior volume of the hygienic article 151. The fold can be formed by an interior or exterior seam, or a crease in the material of the hygienic article. The fold 159 decreases the interior surface area of the hygienic article 151 in contact with the penis, which decreases the amount of force created during use which attempts to remove the hygienic article 151.

In various embodiments, the retaining strap may includes a hook and loop fastening system with either the hook or loop portion affixed to the retaining strap, and the corresponding portion affixed to the tube in an appropriate location. In other embodiments, the retaining strap may be configured to be directly attached to and engage the material of the tube or retaining strap without the need for a separate hook or loop portion affixed to the tube. In such embodiments, the retaining strap may be directly releasably attached to the tube reducing the number of components and manufacturing cost of the hygienic article.

In various embodiments, including those illustrated above, the tube may be be formed from a layered composite material. In some embodiments, the layered composite material includes four identifiable layers, including a multi-layer waterproof outer covering, the absorbent material, an acquisition distribution layer configured to transport urine away from the penis and into the absorbent material, and a topsheet configured to be in contact with a penis. The layers may be combined, and layers may be omitted depending upon the materials selected. For example, a single material may serve as both the top sheet and the acquisition distribution layer. Alternatively, a single material may serve as both the acquisition and distribution layer and the absorbent material. In some embodiments, the multi-layer waterproof outer covering is an elasticized non-woven material, such as a tri-laminate non-woven material.

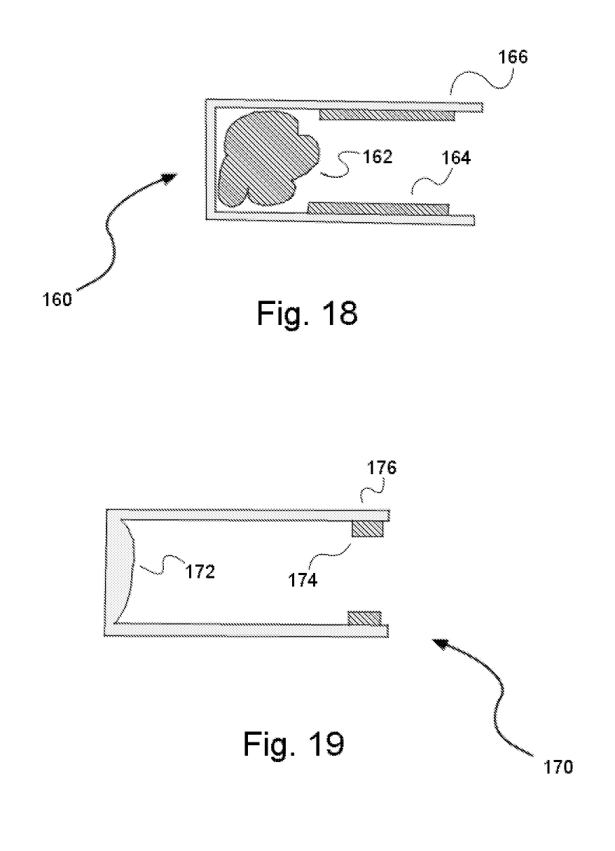

Referring now to FIGS. 18-19, additional embodiments of a hygienic article are illustrated in cross-section. FIG. 18 illustrates a hygienic article 160 with an additional absorbent material 162 in the interior of the hygienic article 160 to aid in absorbing moisture. The additional absorbent material 162 is attached to the wall 166 of the hygienic article 160, or alternatively is placed into the article without any physical attachment. In some embodiments, the hygienic article may also include an additional absorbent pad 164 attached to the wall 166 of the hygienic article 160 between the first and second end.

FIG. 19 illustrates another embodiment of a hygienic article 170 with a gripping material 174 connected to the interior of the wall 176 of the hygienic article 170 near the first end. The gripping material 174 assists in retaining the penis within the hygienic article by way of either mechanical friction or adhesive force. In some embodiments, the hygienic article 170 includes a protrusion 172 formed in the wall 176 of the second end that extends towards the interior of the hygienic article 173. The protrusion 172 reduces minimizes the amount of interior surface that directly contacts the penis and maintains the configuration of the hygienic article to reduce the forces tending to push the hygienic article off the user.

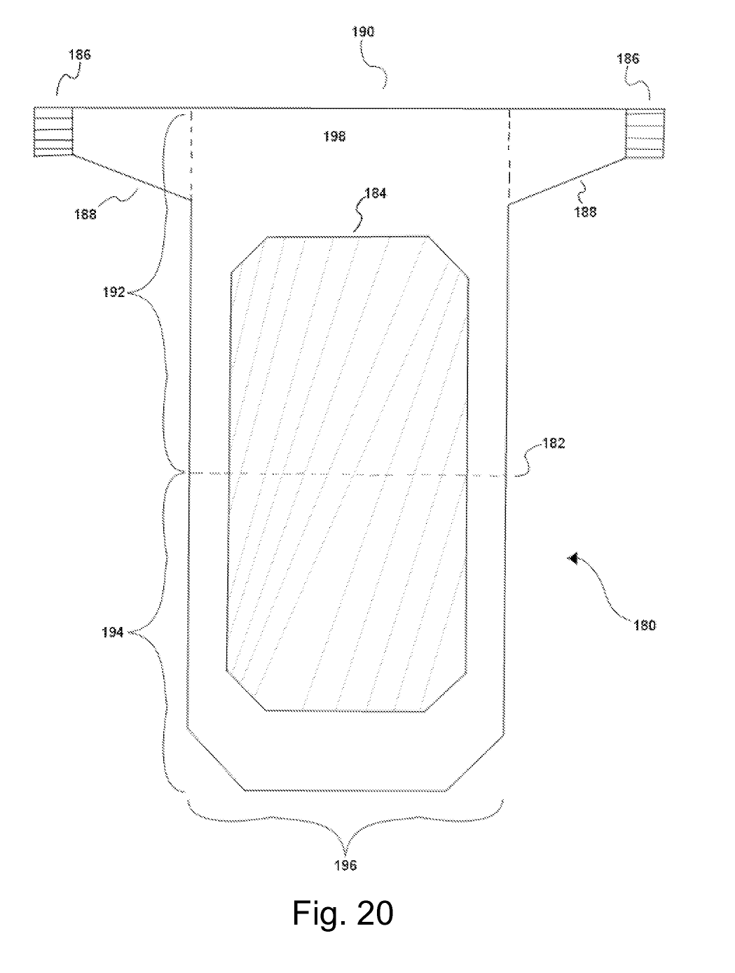

Also disclosed are methods of manufacturing a hygienic article. Referring now to FIG. 20, a hygienic article 180 is illustrated during a manufacturing process. The hygienic article comprises a first end 190, a second end 196, two retaining arms 188, a retaining material 186, an absorbent pad 184, a horizontal fold line 182, a bottom end 192, a top end 194, and a base material 198.

The absorbent pad 184 can be attached to the base material 198 or may simply be placed on the base material 198 without physical attachment. The hygienic article is folded across the horizontal fold line to bring the top end 194 in contact with the bottom end 192, and top end and bottom end are joined along the side portions, such as by welding or sewing to form a finished hygienic article (such as the ones illustrated in FIGS. 12-18). The second end 192 and first end 190 form the opening adapted to receive the penis in the finished hygienic article. As illustrated, the retaining material 186 can be integrally formed with the base material 198 and retaining arms 188, or alternatively may be affixed to the base material during the manufacturing process.

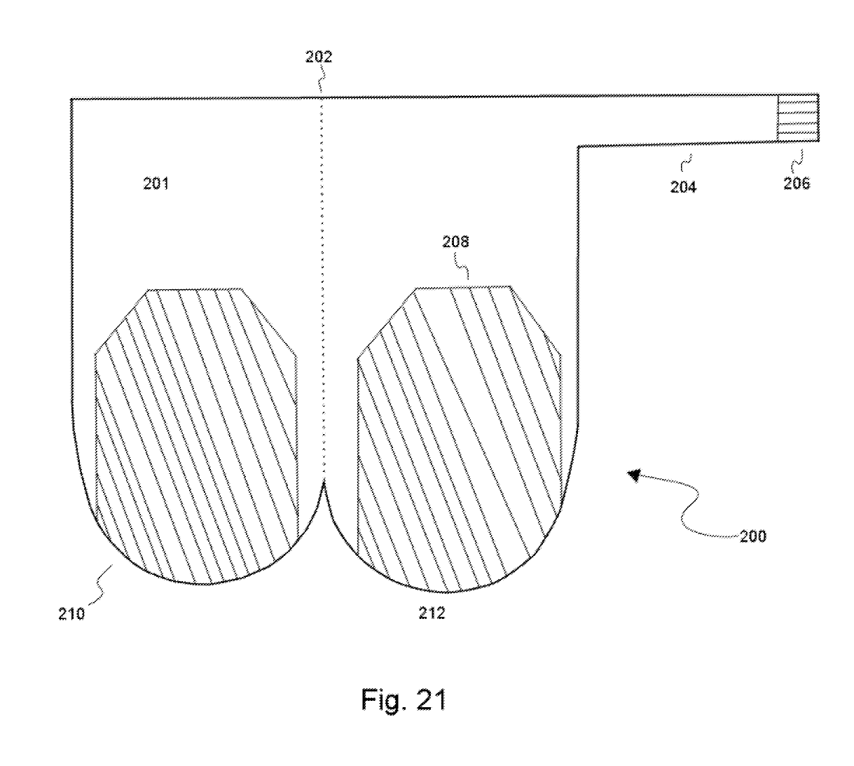

FIG. 21 illustrates a hygienic article 200 unfolded and prior to assembly that includes a base material 201, a vertical folding line 202, a retaining strap 204, a retaining material 206, an absorbent material 208, and a curved end 210.

The hygienic article 200 is folded across the vertical folding line 202 and then mechanically sealed around the resulting edges. This manufacturing process forms the completed hygienic article 200. The absorbent material 208 may be mechanically fixed to the base material 201 in a manner sufficient to prevent the absorbent material from moving during use. Examples of this process include but are not limited to: sewing, ultrasonic welding, heat welding, applying an adhesive material, a hook and loop system, or embedding the pad within the base material 201. In another embodiment, the absorbent material 208 can be placed with the finished article without being attached to the hygienic article.

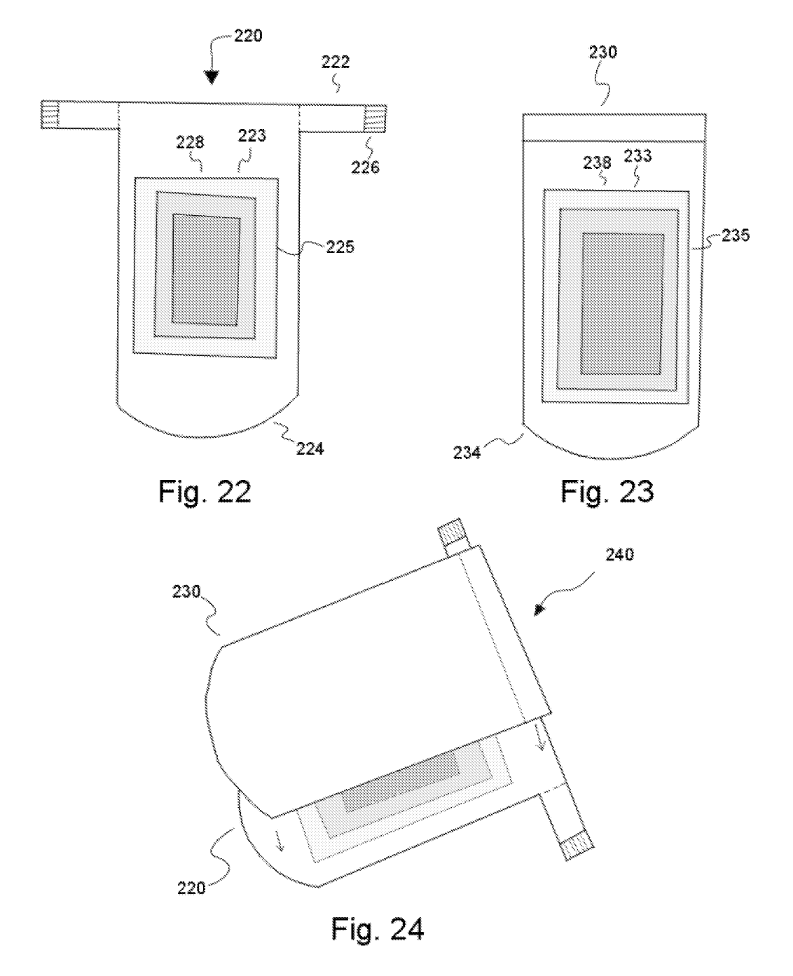

FIGS. 22-24 illustrate the construction of a two-piece hygienic article, comprising a bottom portion 220 and top portion 230, similar to the hygienic article illustrated in FIG. 16.

Referring to FIG. 22, a bottom portion 220 is formed that includes two retaining arms 222, a curved end 224, a retaining material 226, an absorbent pad 228. The bottom portion 220 may optionally include additional layers 223, 225. The absorbent pad 228 and optional additional layers 223, 225 are affixed to the bottom portion 220 or may be loosely placed. Referring to FIG. 23, a top portion 230 is formed that includes an absorbent pad 238, and optionally additional layers 223 and 255, and a curved end 234. The absorbent pad 238 and optional additional layers 233 and 235 are be affixed to the top portion 230 or may be loosely placed. Referring to FIG. 24, the top portion 230 is mated to the bottom portion 220, and then mechanically connected substantially around the edges of the hygienic article.

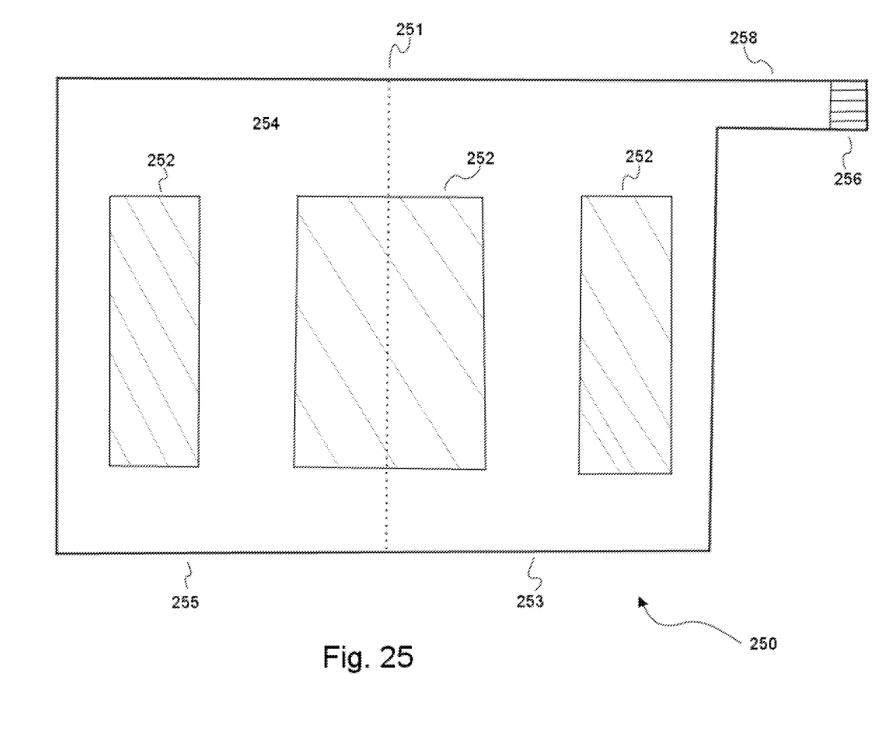

Referring now to FIG. 25, a partially unassembled hygienic article 250 is illustrated that includes a vertical folding line 251, at least one absorbent pad 252, a main body 254, a top portion 255, a bottom portion 253, a retaining material 256, and a retaining arm 258.

The main body 254 is constructed of a fabric material and is substantially square-shaped. The absorbent pads 252 is placed on the main body 254. After folding the main body 254 across the vertical folding line 251, a seam or mechanical bonding method can be used to bond the top portion 255 with the bottom portion 253 forming a finished hygienic article.

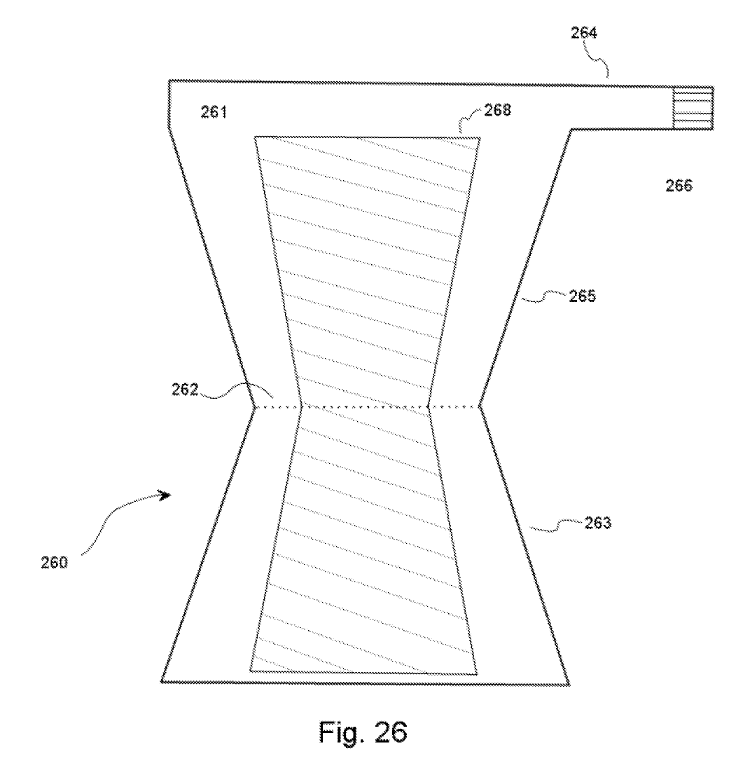

FIG. 26 illustrates another hygienic article 260 prior to assembly. The hygienic article 260 comprises a horizontal folding line 262, a base material 261, a top portion 263, a retaining arm 264, a bottom portion 265, a retaining material 266, and an absorbent pad 268.

The top portion 263 and bottom portion 265 when folded over the horizontal fold line 262 will create a substantially wedge-like shape for the hygienic article 260. The folded hygienic article 260 can then be sewn or mechanically bonded around the non-folded edges to create a finished hygienic article. The retaining arm 264 can be integrally formed with the base material 261 or may be separately attached. In some embodiments, the retaining arm 264 will comprise an elasticized material capable of stretching.

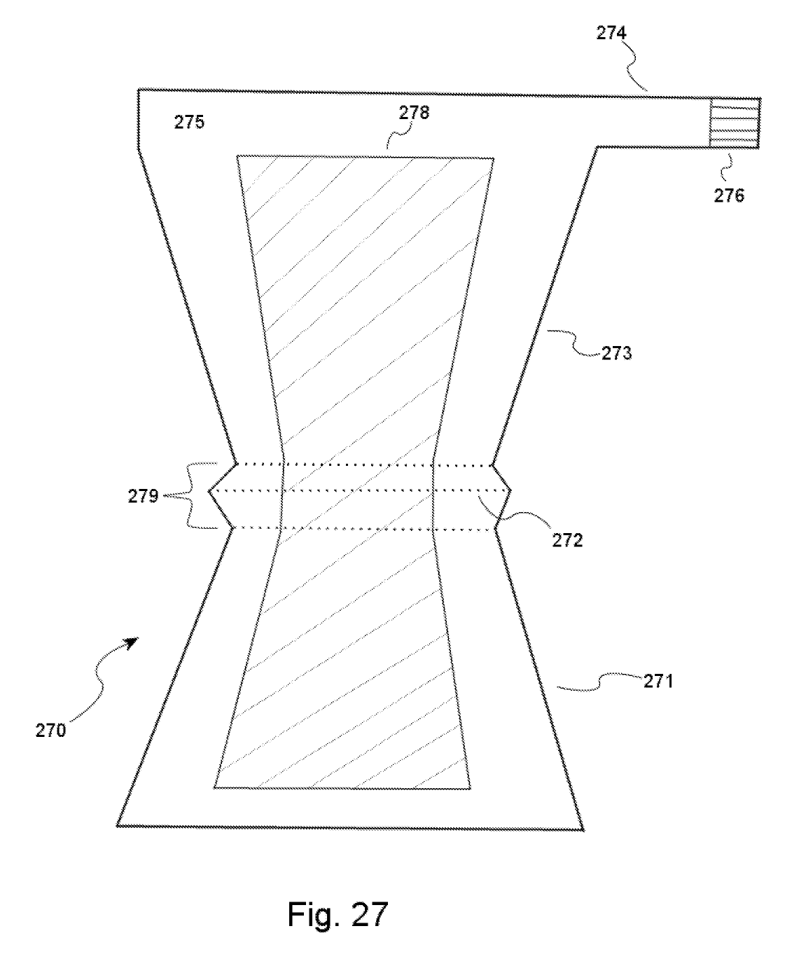

FIG. 27 illustrates an integrally formed hygienic article 270 prior to assembly. The hygienic article 270 comprises a plurality of horizontal folding lines 272, a base material 275, a retaining arm 274, a retaining material 276, an absorbent pad 278, a central fabric section 279, a top portion 271, and a bottom portion 273.

When the top portion 271 is folded over the folding lines 272, the central fabric section 279 will form a second end which may then be folded again to create a folded second end, such as shown in FIG. 18. The top portion 271 and bottom portion 273 are angled so that once they are folded over the folding lines, the hygienic article 270 will be substantially wedge-shaped. The absorbent pad 278 may be attached to the based material 275, or may not be attached and left to nest within the resulting hygienic article 270.

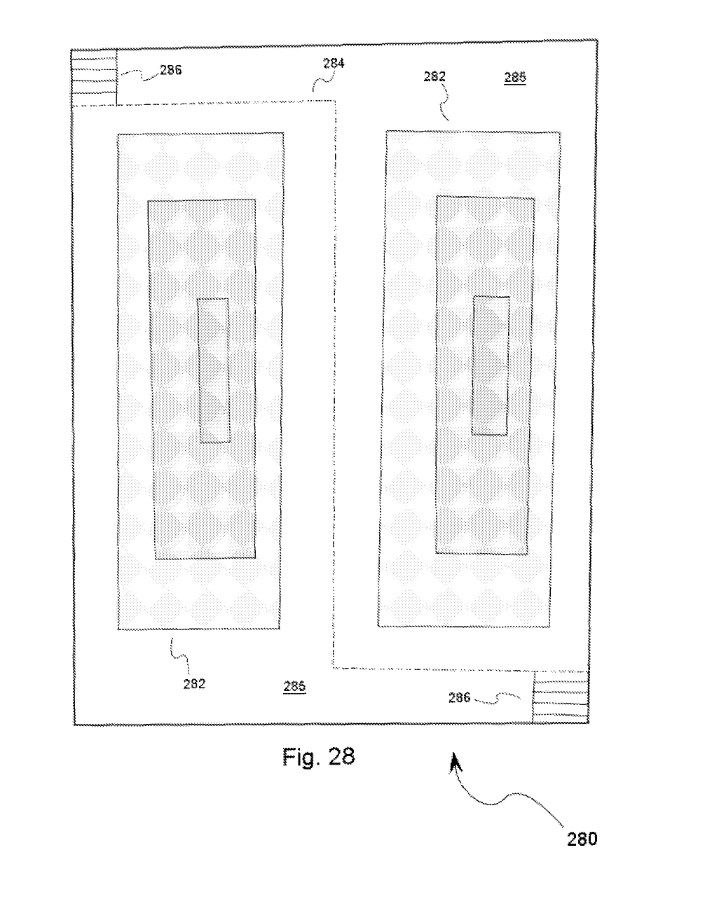

FIG. 28 shows a section of a continuous cutting sheet 280, comprising a cutting line 284, an outline of a single-arm hygienic article 285, a piece of retaining material 286, and an absorbent pad 282. The outline of the single-arm hygienic article 285 and cutting line 284 are arranged on the cutting sheet 280 to enable a continuous line of single-arm hygienic articles 285 to be created without creating waste. The absorbent pad 282 may be multi layered, with each layer being placed on top of one another. The absorbent pad 282 and retaining material 286 can be placed on or attached to the sheet 290 prior to cutting in order to reduce manufacturing costs.

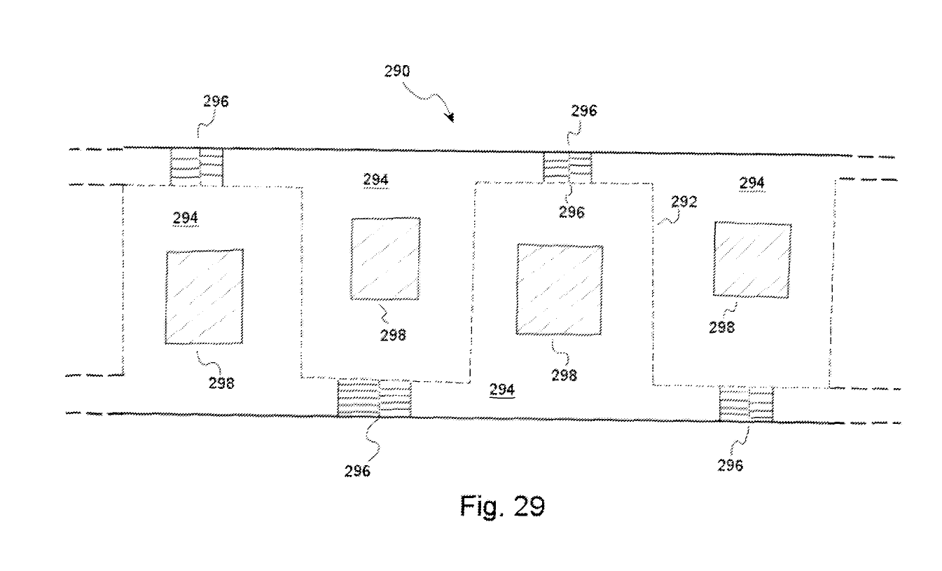

FIG. 29 shows a section of a continuous cutting sheet 290 including a cutting line 292 which is the outline of a two-armed hygienic article 294, a piece of retaining material 296, and an absorbent pad 298. The two-arm hygienic article 294 and the cutting line 292 are arranged on the cutting sheet 290 to enable a continuous line of two-arm hygienic articles 294 to be created without creating waste. The absorbent pad 298 and retaining material 296 can be placed on or attached to the sheet 290 prior to cutting in order to reduce manufacturing costs.

As used herein, the term “tube” means a structure with a closed cross sectional area and at least one open end. The cross-sectional area may circular, square, rounded or have some other shape.

While certain embodiments have been described, it must be understood that various changes may be made and equivalents may be substituted without departing from the spirit or scope of the present disclosure. Within the embodiments described, it is also to be understood that the features and characteristics disclosed are interchangeable with other embodiments. In addition, many modifications may be made to adapt a particular situation or material to the teachings of the disclosure without departing from its spirit or scope.

What is claimed is:

1. A hygienic article for a male comprising:
a water resistant tube having an open first end and a closed second end, wherein the first end defines an opening configured to receive a limp penis within an interior of the tube, and
an absorbent material disposed within a portion of the interior of the tube configured to absorb urine,
wherein the second end is configured to provide radial support to the tube to reduce axial force tending to remove the hygienic article from the penis.

2. The hygienic article for a male as claimed in claim 1, wherein the tube comprises a layered composite material.

3. The hygienic article for a male as claimed in claim 2, wherein the layered composite material comprises a multi-layer waterproof outer covering, the absorbent material, an acquisition distribution layer configured to transport urine away from the penis and into the absorbent material, and a topsheet configured to be in contact with a penis.

4. The hygienic article for a male as claimed in claim 3, wherein the multi-layer waterproof outer covering is an elasticized non-woven material.

5. The hygienic article for a male as claimed in claim 1, wherein the second end comprises at least two flaps, wherein the at least two flaps are integral with the tube, folded and physically connected to each other to form the closed second end of the tube,

6. The hygienic article for a male as claimed in claim 1, wherein the second end comprises pleating.

7. The hygienic article for a male as claimed in claim 6, wherein the pleating is a box pleat.

8. The hygienic article for a male as claimed in claim 1, wherein the second end further comprises an external flap configured to provide structural support to maintain the shape of the second end while the hygienic article is in use.

9. The hygienic article for a male as claimed in claim 8, wherein the exterior flap is perpendicular to a longitudinal axis extending between the first end and the second end of the tube.

10. The hygienic article for a male as claimed in claim 1, wherein the second end is integrally formed with the tube, and the second end further comprises a semi-circular cross section.

11. The hygienic article for a male as claimed in claim 1, wherein the second end further comprises an interior fold.

12. The hygienic article for a male as claimed in claim 11, wherein the tube further comprises two side portions extending between the first end and the second end, and the two side portions each comprise an interior fold along at least a portion of the length of the side portions.

13. The hygienic article for a male as claimed in claim 1, wherein the tube is formed from two separate pieces of layered material physically connected around at least a portion of the perimeter of the pieces to form the hygienic article.

14. The hygienic article for a male as claimed in claim 1, wherein the tube has a rectangular cross section.

15. The hygienic article for a male as claimed in claim 1, wherein the water resistant tube further comprises a top portion, a bottom portion, and at least two side portions, in which
the top portion and the bottom portion are parallel with respect to each other,
the at least two side portions are parallel with respect to each other,
and the first end and second end are positioned parallel with respect to each other.

16. The hygienic article for a male as claimed in claim 1 further comprising at least one retaining strap located adjacent the first end.

17. The hygienic article for a male as claimed in claim 16, wherein the retaining strap further comprises a retaining material, wherein the retaining material can be directly releasably attached to the tube.

18. The hygienic article for a male as claimed in claim 1, further comprising at least two side portions, in which
the at least two side portions are parallel with respect to each other, and
the second end is positioned perpendicular to the at least two side portions.
